(19) United States
(12) Patent Application Publication
(10) Pub. No.: US 2009/0247450 A1
(43) Pub. Date: Oct. 1, 2009

(54) G-CSF LIQUID FORMULATION

(76) Inventor: Michael Mack, Bad Vilbel (DE)

Correspondence Address: KNOBBE MARTENS OLSON & BEAR LLP, 2040 MAIN STREET, FOURTEENTH FLOOR, IRVINE, CA, 92614, US

(21) Appl. No.: 12/281,130

(22) PCT Filed: Mar. 1, 2007
(86) PCT No.: PCT/EP2007/051947

§ 371 Date: Apr. 6, 2009

(30) Foreign Application Priority Data
Mar. 1, 2006 (DE) 10 2006 009 437.9

(51) Int. Cl. A61K 38/18; A61P 43/00

(52) U.S. Cl. 514/8

(57) ABSTRACT

The invention relates to G-CSF liquid formulations that have a long shelf-life and to methods for producing the same. The invention also relates to liquid formulations containing G-CSF as the active substance, acetate as the buffer substance, polysorbate 20 or polysorbate 80 as the surfactant and optionally pharmaceutically acceptable adjuvants, the formulations having a pH between 4.1 and 4.4.

The present invention relates to storage-stable liquid formulations of G-CSF as well as to methods for their production. In particular, the present invention relates to liquid formulations containing G-CSF as active agent, acetate as buffer substance, polysorbate 20 or polysorbate 80 as tenside and optionally pharmaceutically acceptable excipients, wherein the formulations have a pH value in the range of between 4.1 to 4.4.

G-CSF (granulocyte colony stimulating factor) is a naturally occurring growth factor belonging, in a broader sense, to the family of cytokines and, more precisely, to the group of colony stimulating factors. G-CSF plays a decisive role in hematopoiesis and enhances the proliferation and differentiation of hematopoietic precursor cells and the activation of neutrophils. It is due to said characteristics that G-CSF is applied in various medical areas, like for example in the reconstitution of normal blood cell populations subsequent to chemotherapy or irradiation or for stimulating the immune response towards infectious pathogens. In clinical practice, G-CSF is thus mainly applied in tumor prevention and, in particular, in the treatment of neutropenia as a consequence of chemotherapy and furthermore in the course of bone marrow transplantations and in the treatment of infectious diseases.

The recombinant production of G-CSF was first described in patent literature in WO-A-87/01132 in 1987. The first commercial G-CSF preparation on the basis of recombinant G-CSF was approved in Germany in 1991 and is produced and distributed by Amgen under the trade name Neupogen®.

According to the German physicians desk reference ROTE LISTE 2005, the product Neupogen® (prefilled syringes) consists of the following components: G-CSF at a concentration of 600 μg/ml or 960 μg/ml, sodium acetate, sorbitol, polysorbate 80 and water.

In the art, various patent documents deal with pharmaceutical preparations of G-CSF. In EP-A-0 373 679, formulations of G-CSF are described, in which the protein is stabilized by the presence of an acid, an acidic pH value and a low conductivity of the formulation.

DE-A-37 23 781 relates generally to the use of G-CSF in combination with a pharmaceutically acceptable surface-active agent, saccharide, protein or a pharmaceutically acceptable high molecular compound.

In WO-A-94/14465, the use of maltose, cellobiose, gentiobiose, isomaltose, raffinose, sucrose and further sugars for stabilizing preparations containing G-CSF is described.

In WO-A-94/14466, preparations containing G-CSF are disclosed which contain an amount of tenside that is smaller than the amount of G-CSF employed, as well as a buffer substance.

WO-A-93/03744 describes formulations containing G-CSF which contain a preservative that is chlorobutanol, benzyl alcohol, or benzalkonium.

EP-A-0 988 861 discloses formulations containing G-CSF which contain HEPES, TES, or tricine as buffer substance.

WO-A1-2005/042024 discloses pharmaceutical preparations of G-CSF having a pH value of more than 4.0 which contain an acid and are free of tensides.

It is the problem underlying the present invention to produce a G-CSF preparation that can be stored for longer time periods in liquid form and that does not require stabilizing additives like HSA, amino acids or preservatives. In this way, a storage-stable G-CSF liquid formulation is to be provided that preferably avoids any risk with respect to the tolerability of the formulation.

According to the present invention, this problem is solved by means of the subject of claim 1. Preferred embodiments are defined in the dependent claims.

It was found that, even in the absence of HSA, amino acids, or polymeric stabilizers, liquid G-CSF compositions containing acetate as buffer substance and polysorbate 20 and/or polysorbate 80 as tenside and having a pH value in the range of between 4.1 to 4.4 can be stored in liquid form for longer time periods without significant losses in stability.

Thus, the present invention relates to a storage-stable aqueous liquid formulation of G-CSF comprising, besides recombinant human G-CSF as active agent, acetate as buffer and polysorbate 20 (polyoxyethylene sorbitan monolaurate, also referred to as Tween 20) or polysorbate 80 (polyoxyethylene sorbitan monooleate, also referred to as Tween 80) or a mixture thereof as tenside and having a pH value in the range of between 4.1 to 4.4.

In a preferred embodiment, the tenside is polysorbate 20.

However, it was also found that further polyoxyethylene sorbitan alkyl esters are also suitable for the use as detergent in the formulations of the present invention.

In general, the tenside is contained at a concentration in the range of between 0.0005% (w/v) to 0.05% (w/v), preferably in the range of between 0.001% (w/v) to 0.01%, particularly preferably in the range of between 0.002% (w/v) to 0.008%, and most preferably in the range of between 0.004% (w/v) to 0.006% (w/v), in relation to the total volume of the solution. Normally, the formulations according to the present invention contain the tenside polysorbate 20 and/or 80 at a concentration of 0.004% (w/v), 0.005% (w/v), or 0.006% (w/v).

The concentration of the buffer substance acetate is preferably selected in such a way that, with the pH value in the range of between 4.1 to 4.4 according to the present invention, both the pH-stabilizing effect and a sufficient buffer capacity are achieved, while simultaneously the ionic concentration and thus the conductivity are kept as low as possible in order to avoid aggregate formation. In general, acetate is contained at a concentration in the range of between 0.5 to 150 mmol/l, preferably in the range of between 1 to 100 mmol/l, particularly preferably in the range of between 2 to 50 mmol/l, and most preferably between 5 and 20 mmol/l. Normally, the concentration of the acetate is 10 mmol/l.

The buffer substance acetate can be employed both in form of the free acid and in form of the salt. As salts, in particular the physiologically acceptable salts are employed, for example alkali or ammonium salts, preferably the sodium salt.

The pH value of the formulation lies within a range from more than 4.0 to 4.5, in particular between 4.1 and 4.4, preferably between 4.1 and 4.3 or between 4.15 and 4.35, particularly preferably between 4.2 and 4.3 or between 4.25 and 4.35, also particularly preferably between 4.25 and 4.3, and most preferably at about 4.25 or about 4.3.

If desired, the pH value of the composition can additionally be adjusted to a pH value of more than 4.0 to 4.5, from 4.1 to 4.4, from 4.1 to 4.3, from 4.15 to 4.35, from 4.2 to 4.3 or from 4.25 to 4.35, from 4.25 to 4.3 or to about 4.25 or about 4.3 with the aid of further acids or bases. Suitable acids are, for example, hydrochloric acid, phosphoric acid, citric acid and sodium or potassium hydrogen phosphate. Suitable bases are, for example, alkali and alkaline earth hydroxides, alkali carbonates, alkali acetates, alkali citrates and dialkali hydrogen phosphates, for example sodium hydroxide, sodium acetate, sodium carbonate, sodium citrate, disodium and dipotassium hydrogen phosphate and ammonia.

Preferably, the pH value is adjusted by means of NaOH. In one embodiment, as a consequence of adjusting the pH value with NaOH, the formulation of the present invention thus further contains sodium ions.

In one embodiment, the formulation further contains a polyol as pharmaceutically acceptable additive, in particular a sugar alcohol, which particularly preferably is mannitol or sorbitol. Said additives are particularly suitable as isotonizing agents for making the compositions of the present invention isotonic with the patient's blood.

Normally, the concentration of the polyol is up to 10.0% (w/v) in relation to the total volume of the composition. Preferably, the concentration is up to 8.0% (w/v), particularly preferably up to 6.0% (w/v). Particularly preferably, sorbitol or mannitol is contained at a concentration of 5.0% (w/v).

The concentration of G-CSF depends on the respectively desired concentration of the agent in the prefilled syringe, in which the liquid formulation according to the present invention is stored and eventually applied. The trade product Neupogen® (ROTE LISTE 2005, No. 51 038) is available, for example, in the following concentrations: 300 μg/0.5 ml; 480 μg/0.5 ml and 300 μg/1.0 ml. Herein, 10 μg protein correspond to about 1.0 million International Units (IU). In the case of the trade product Granocyte (ROTE LISTE No. 51 036), the activity of the recombinant G-CSF is slightly higher; here, 10 μg protein correspond to an activity of 1.28 million IU.

Within the scope of the present invention, typical G-CSF concentrations are between 0.01 and 3.0 mg/ml, preferably between 0.1 and 2.5 mg/ml, particularly preferably in a range of between 0.5 and 2.0 mg/ml, and most preferably between 0.6 and 1.5 mg/ml. The concentrations 0.6 mg/ml and 0.96 mg/ml represent preferred embodiments. Here, the activity of the employed G-CSF normally is about 1.0±0.6×10⁸ units/mg.

In more highly concentrated starting solutions, often referred to as bulk solutions, the agent concentration can also be higher; potentially 5 μg/ml and more.

The compositions according to the present invention can contain further conventional, in particular physiologically acceptable, stabilizers and/or excipients or additives. For example further tensides, isotonizing agents, reducing agents, antioxidants, complexing agents, cosolvents, diluents and chaotropic agents.

It is, however, preferred that the formulation does not contain any polymeric stabilizers. Thus, for example polyalkylene glycols like polyethylene glycol, hydroxyethyl starch, dextrans, cyclodextrins, but also proteins like HSA and other plasma proteins or gelatin are to be omitted.

For injection purposes, the use of pure water is preferred. However, further conventional solvents that are suitable for pharmaceutical preparations can also be employed. Solvents like glycerol, polyethylene glycol and propylene glycol are preferably omitted, however.

The employment of buffer substances like tartrate, succinate, HEPES, TES and tricine is also omitted.

Preferably, amino acid stabilizers are also omitted.

On the whole, it is preferred to keep the number of different excipients in the formulation as low as possible. Correspondingly, sugars other than mannitol or sorbitol are preferably avoided and the buffer substances contained in the formulation are preferably narrowed down to acetate exclusively.

The components of the formulation can be obtained from conventional sources, for example from the company Sigma or the company Merck.

The recombinant G-CSF can be produced and purified according to state-of-the-art protocols.

The G-CSF is biologically active G-CSF that is capable of enhancing the differentiation and proliferation of hematopoietic precursor cells and of effecting the activation of mature cells of the hematopoietic system. Thus, the G-CSF formulation is suitable for treating indications in cases where the administration of G-CSF is advantageous. It is understood that the term “biologically active human G-CSF” also includes mutants and modifications of G-CSF, whose amino acid sequence is altered as compared to the wild type sequence, but which have a biological activity similar to that of the wild type G-CSF, like for example those described in WO 01/87925 and EP 0 456 200. G-CSF in the sense of the present invention also denotes G-CSF conjugates in which the protein is present in conjugated form, for example with polymers like for example polyalkylene glycols, in particular with polyethylene glycol, as so-called PEGylated G-CSF or PEG-G-CSF, or hydroxyalkyl starches, in particular with hydroxyethyl starch. The G-CSF can be glycosylated or non-glycosylated. While the recombinant protein that is produced in E. coli has no carbohydrate structures and is expressed with an N-terminal methionine residue, the G-CSF that is produced in eukaryotic cells, like for example CHO cells, is normally glycosylated.

Preferably, the G-CSF contained in the liquid formulation is human Met-G-CSF, produced in E. coli cells. Various expression systems are commercially available for the expression in E. coli cells. Suitable, for example, is the expression of human G-CSF under the control of an inducible promoter, for instance an IPTG-inducible promoter. See, for example, Sambrook and Russel, Molecular Cloning—A Laboratory Manual, 3^{rd} edition 2001, Cold Spring Harbor Laboratory Press, Cold Spring Harbor, N.Y., chapter 15, or established manufacturers' protocols, for example by Promega or Stratagene.

The fermentation of the host cells can be conducted according to standard protocols, like they are described in patent and scientific literature, as can the subsequent purification including harvesting the so-called inclusion bodies, which contain the G-CSF that is overexpressed in E. coli, lysing, solubilizing, refolding, and chromatographically purifying said inclusion bodies; suitable protocols can be found both in patent literature and in standard references of protein chemistry as well as in laboratory manuals.

The isolation and purification of G-CSF, including solubilizing and refolding, are described, for example, in EP-A-0 719 860. General techniques for solubilizing and renaturing denatured proteins have been described in EP-A-0 512 097, EP-A-0 364 926, EP-A-0 219 874, and WO 01/87925 and can moreover be taken from scientific literature and standard references of protein chemistry.

The refolded protein is subsequently purified by means of chromatographic methods, i.e. it is separated from other proteins and further contaminations that are contained after solubilizing and renaturing.

Among others, patent document WO 87/01132 A1, which was already referred to above and in which the production of G-CSF in E. coli host cells was described for the first time, deals with chromatographic purification. In the context of purifying the recombinant G-CSF, a cation exchange chromatography using a CM cellulose column is conducted in Example 7 in WO 87/01132 A1.

In EP 0 719 860 A1, the G-CSF is purified subsequently to solubilization and oxidation, the solubilizer is removed by Dowex treatment, followed by an anion exchange chromatography and a cation exchange chromatography. In EP 0 719 860 A1, CM Sepharose is also used for cation exchange chromatography.

In WO 03/051922 A1, a purification method for G-CSF is described, in which a metal affinity chromatography is conducted, more precisely an immobilized metal affinity chromatography (IMAC). Subsequently to the metal affinity chromatography, a cation exchange chromatography and/or a gel filtration can be conducted in WO 03/051922.

In WO 01/04154 A1, a method for purifying G-CSF is described, in which initially a hydrophobic interaction chromatography and subsequently thereto a hydroxyapatite chromatography are conducted. Subsequently to the hydroxyapatite chromatography a cation exchange chromatography is performed.

The purity of the G-CSF that is formulated in the formulation of the present invention should amount to at least 95%, preferably to at least 97%, particularly preferably to at least 99% and most preferably to more than 99%. Here, purity can be checked by means of HPLC analyses. Suitable therefore are rp-SEC and IEX analyses. The person skilled in the art can take suitable materials and protocols for conducting the rpHPLC or the SEC-HPLC from product information provided by suppliers like Vydac (http://www.vydac.com) or TOSOH Bioscience (http://www.tosohbiosep.de).

In the preparations according to the present invention, G-CSF with a purity of more than 99%, preferably more than 99.5%, is normally employed.

Preferably, the activity of the employed G-CSF should not be lower than 50,000 IU/μg; a G-CSF having an activity 80,000 IU/μg is particularly suitable; most suitable is a G-CSF having an activity of about 100,000 IU/μg or more.

Determining the yield of Met-G-CSF is also described in Herman et al. (1996) Pharm. Biotechnol. 9: 303-328. Here, the exact ratio of Met-G-CSF is determined by means of integrating the peak area and conversion on the basis of the extinction coefficient.

Subsequent to purification, the G-CSF can be analyzed with respect to its amount and activity. A qualitative analysis can be conducted via an SDS-PAGE followed by Coomassie Brilliant Blue staining or via an rpHPLC. A commercially available G-CSF preparation can be used as a standard for the analyses. In addition, a peptide map or a mass spectroscopy can be performed. The activity of the purified G-CSF can be determined by means of different biological test methods, like they are described, for example, in Shirafuji et al. (1989) Exp. Hematol. 17(2): 116-119; Oh-Eda et al. (1990) J. Biol. Chem. 265(20): 11432-11435; Stute et al. (1992) Blood 79(11): 2849-2854 and Oshima et al. (2000) Biochem. Biophys. Res. Commun. 267(3): 924-927.

Incidentally, all the chromatographies are conducted according to the recommendations and protocols of the suppliers of the matrices or columns (for example with respect to flow rate, column volumes employed for washing or for elution, diameters and bed heights of the columns, etc.).

The biological activity of the obtained recombinant G-CSF can be determined by means of a bioassay and can be compared to the biological activity of a standard, i.e. of commercially available G-CSF (Neupogen®). To this end, the murine cell line NFS-60, which is responsive to G-CSF, can be used. For this purpose, said cell line is cultivated in RPMI 1640 medium (Bachem, Heidelberg, Germany) that contains 1.5 g/l sodium carbonate, 4.5 g/l glucose, 10 mM Hepes and 1.0 mM sodium pyruvate and is supplemented with 2 mM glutamine, 10% FCS, 0.05 mM 2-mercaptoethanol and 60 ng/ml G-CSF.

For the activity test, the cells are washed twice with G-CSF-free medium, seeded in 96-well plates at a concentration of 2×10⁴ cells per well and incubated for three days at 37° C. and 4.5% CO₂ with different concentrations of the purified G-CSF and of the standard. Subsequently, the cells are stained with XTT Reagent and absorption is measured at 450 nm in a microtiter plate reader. It is desired that the cells treated with the obtained G-CSF grow as well as the cells treated with the standard (for example the trade product Neupogen®), as in this case an equal biological activity of the two G-CSF samples can be assumed.

The production of the formulation is also conducted according to methods conventional in the art.

Conventionally, the buffer and tenside components and, optionally, further pharmaceutically acceptable excipients are first dissolved at suitable amounts in the aqueous solvent, normally in sterile water. If necessary, the pH value is adjusted by means of acetate solution or with other acids or bases, like the exemplary ones mentioned above. Subsequently to a conventional sterilization step, for example filtration through a sterile filter, G-CSF is added at the desired concentration. However, it is also easily possible to provide G-CSF in an aqueous solution and to subsequently adjust the pH with acetate and/or suitable acids or bases, preferably NaOH, to the desired value.

Eventually, the prepared liquid formulation is filled into a suitable receptacle, where it is stored until application. In particular, the receptacle is a prefilled syringe, a vial or an ampoule.

The compositions according to the present invention can be employed in the various forms of administration. The formulations according to the present invention are, for example, suitable as injection or infusion solutions, in particular for intravenous, intramuscular, or subcutaneous administration. However, the compositions can also be used for producing other forms of administration, for example transfersomes, liposomes or hydrogels.

The liquid formulation according to the present invention does not only provide the advantages of omitting potentially immunogenic compounds and altogether containing a minimum possible number of ingredients, it also does not require lyophilization in any phase of the production process. Thus, the expenses related to lyophilization are saved on the one hand, and the risks of mechanical problems, for example if the lyophilisate cannot be reconstituted in a complete or sufficient manner, are avoided on the other hand.

Within the scope of the present invention, the term “storage-stable” is understood to denote that the content of active G-CSF molecules is still 80% or more of the initial concentration after storing the G-CSF liquid formulation for three months at 25° C. Preferably, the residual content of G-CSF activity is still at least 85%, more preferably at least 90%, and most preferably at least 95% of the initial activity after three months of storage at 25° C. The activity of the G-CSF can be determined by means of conventional activity tests, like already described for G-CSF in the art. Within the scope of the present invention, the term “liquid formulation” is understood to denote that the formulation of G-CSF, together with further substances contained in the formulation, is not lyophilized in any phase of the production process, i.e. neither before nor during nor subsequently to mixing the substances, and that the formulation is intended for intravenous or subcutaneous application as injection solution or infusion solution.

In a preferred embodiment, the formulation contains no further ingredients other than the agent G-CSF, a tenside selected from polysorbate 20, polysorbate 80 or a mixture thereof, acetate, sorbitol, sodium ions, and water and has a pH value in the range of between 4.2 to 4.3 or in the range of between 4.25 to 4.35, in particular in the range of between 4.25 to 4.3. The use of polysorbate 20 as the only tenside in the formulation is particularly preferred.

### EXAMPLES

The following Examples are employed to illustrate the invention without limiting it thereby.

Compositions containing G-CSF were prepared by first dissolving the buffer substance acetate in form of the sodium salt together with polysorbate 20 or polysorbate 80 and sorbitol in distilled and sterile water and subsequently adjusting the pH value with NaOH to the desired value between 4.2 and 4.3. Non-glycosylated recombinant human G-CSF (Filgrastim, Met-G-CSF) was added at the desired concentration. Preparing and filling the formulation in prefilled syringes was preferably conducted in a nitrogen atmosphere.

The specific formulations of the compositions prepared according to the present invention as well as their pH values are given in the following tables. The use of polysorbate 80 instead of polysorbate 20 led to comparable results. The same applies to the use of mannitol instead of sorbitol.

| Ingredient | Formul. 1 | Formul. 2 | Formul. 3 | Formul. 4 | Formul. 5 | Formul. 6 |
|---|---|---|---|---|---|---|
| G-CSF | 0.6 | 0.6 | 0.6 | 0.96 | 0.96 | 0.96 |
| mg/ml | | | | | | |
| Sorbitol | 5.0 | 5.0 | 5.0 | 5.0 | 5.0 | 5.0 |
| %, w/v | | | | | | |
| Acetate buffer | 10 | 10 | 10 | 10 | 10 | 10 |
| mM | | | | | | |
| Polysorbate 20 | 0.004 | 0.004 | 0.004 | 0.004 | 0.004 | 0.004 |
| %, w/v | | | | | | |
| NaOH 0.1 n* | q.s. | q.s. | q.s. | q.s. | q.s. | q.s. |
| Water | 1.0 ml | ad 1.0 ml | ad 1.0 ml | ad 1.0 ml | ad 1.0 ml | ad 1.0 ml |
| pH | 4.2 | 4.25 | 4.3 | 4.2 | 4.25 | 4.3 |

*for adjusting the pH value

The formulations according to the present invention were stored at different temperatures for different periods of time together with corresponding formulations having a pH value of 4.0, which represent the state of the art and served as comparison formulations.

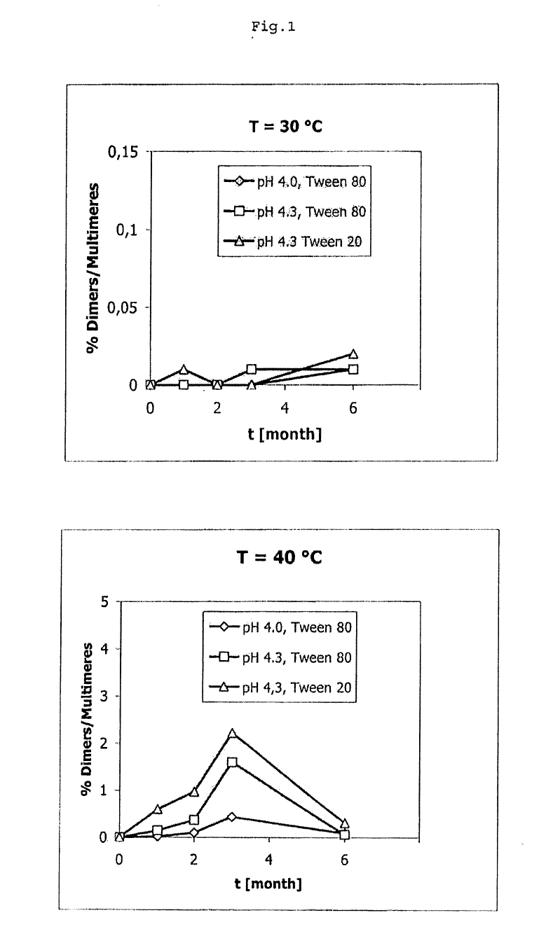

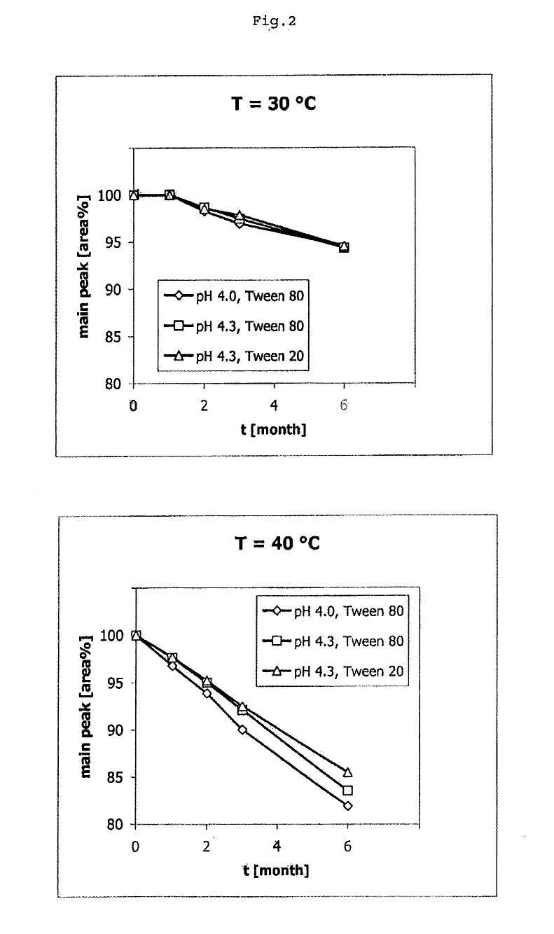

Some data from long-term stability analyses are presented in the appended Figures (G-CSF concentration 0.6 mg/ml in each case). FIG. 1 shows SEC analyses and FIG. 2 shows IEX analyses, in each case with a storage time of up to 6 months at 30° C. and 40° C., respectively.

All in all, the formulations according to the present invention showed results comparable to those of the comparison formulation, which only differed from the tested formulations according to the present invention by exhibiting a more acidic pH value, i.e. pH 4.0.

1. A liquid formulation of G-CSF, comprising G-CSF as an active ingredient, acetate as a buffer, Polysorbate 20 and/or Polysorbate 80 as a surfactant and optionally pharmaceutically acceptable excipients, wherein the formulation has a pH value in the range of between 4.1 to 4.4.

2. The liquid formulation of claim 1, wherein the pH value is between 4.1 to 4.3.

3. The liquid formulation of claim 2, wherein the pH value is between 4.2 to 4.3.

4. The liquid formulation of claim 3, wherein the pH value is 4.25.

5. The liquid formulation of claim 1, wherein the concentration of acetate buffer is between 2 and 50 mmol/l.

6. The liquid formulation of claim 5, wherein the concentration of acetate buffer is 10 mmol/l.

7. The liquid formulation of claim 1, wherein the formulation comprises sorbitol and/or mannitol as a pharmaceutically acceptable excipient.

8. The liquid formulation of claim 7, wherein the formulation contains comprises sorbitol.

9. The liquid formulation of claim 1, wherein the formulation does not contain a preservative.

10. The liquid formulation of claim 1, wherein the formulation does not contain amino acids.

11. The liquid formulation of claim 1, wherein the formulation does not contain polymeric stabilizing agents.

12. The liquid formulation of claim 1, wherein the pH value is adjusted with NaOH.

13. The liquid formulation of claim 1, further comprising sodium ions.

14. The liquid formulation of claim 1, consisting essentially of G-CSF, Polysorbate 20 and/or Polysorbate 80, sorbitol, acetate as a buffer and sodium.

15. The liquid formulation of claim 1, wherein the surfactant is Polysorbate 20.

16. A method for producing the liquid formulation of G-CSF according to claim 1, comprising mixing the G-CSF with a solution comprising the acetate buffer, the Polysorbate 20 and/or Polysorbate 80 surfactant and the optionally pharmaceutically acceptable excipients.
